(19) United States
(12) Patent Application Publication
(10) Pub. No.: US 2025/0121025 A1
(43) Pub. Date: Apr. 17, 2025

(54) SCALP-DERIVED PEPTIDES ALLEVIATING HAIR LOSS SYMPTOMS

(71) Applicant: NEWTREE CO., LTD, Seoul (KR)

(72) Inventors: Dong Woo Lee, Seoul (KR); Jae Eun Lee, Seoul (KR); Jung Mo Lee, Seoul (KR); Ji Young Lee, Seoul (KR)

(73) Assignee: NEWTREE CO., LTD, Seoul (KR)

(21) Appl. No.: 18/442,781

(22) Filed: Feb. 15, 2024

(30) Foreign Application Priority Data
Oct. 16, 2023 (KR) 10-2023-0138088

(51) Int. Cl. A61K 38/07; A61K 8/64; A61P 17/14; A61Q 7/02

(52) U.S. Cl. CPC A61K 38/07; A61K 8/64; A61P 17/14; A61Q 7/02

(57) ABSTRACT

The present invention relates to a novel scalp-derived peptide having the ability to alleviate alopecia symptoms, and since the peptide has an activity capable of promoting or inducing the proliferation or activity of cells associated with hair formation, a composition including the peptide can alleviate the progression of alopecia and promote the nourishment and growth of hair, and thus is effective for preventing, treating or ameliorating alopecia.

### CROSS-REFERENCE TO RELATED APPLICATIONS

This application claims priority to Korean Patent Application No. 10-2023-0138088 (filed on Oct. 16, 2023), which is hereby incorporated by reference in its entirety.

### SEQUENCE LISTING

This application contains a Sequence Listing submitted via XML file and hereby incorporated by reference in its entirety. The sequence listing is named “432-0328_Sequence_Listing.xml”, created on Feb. 15, 2024 and 2,988 bytes in size.

### BACKGROUND

The present invention relates to a novel scalp-derived peptide having the ability to alleviate alopecia symptoms and a use thereof.

The human body has approximately 100,000 to 150,000 hairs on average; as the cell division and activity of hair follicle is interrupted during the process of repeating the anagen, catagen, and telogen phases, existing hair is pushed out by newly growing hair and falls out naturally. This natural hair loss phenomenon occurs with an average of 100 hairs falling out per day, but alopecia occurs as a type of disease when a greater amount of hair falls out than the natural hair loss described above, or when hair is not present or does not grow in areas where it should be normally present. Although hair loss may occur in the natural aging process, it may also be caused by external environmental factors such as hormonal changes, stress, air pollution, and processed foods.

Minoxidil and finasteride are representative drugs used to promote hair growth. Minoxidil may cause side effects such as edema, arrhythmia, and hair growth in unwanted areas when applied long-term, and it is known that the effects of minoxidil are most pronounced between 6 months and 1 year after use, and the effects gradually decrease after that period. Further, finasteride is known as a substance that suppresses the activity of 5-α-reductase, an enzyme that is involved in the metabolism of the male hormone testosterone in hair follicles, but it has also been reported to cause increases in sexual dysfunction, depression, and suicidal impulses. Furthermore, since finasteride increases the possibility of giving birth to a deformed child, it cannot be applied to women of childbearing age or pregnant women. In addition, it is known that when the application of either drug is discontinued, alopecia progresses again. In addition to these drugs, there is valproic acid, but it is known that taking valproic acid during pregnancy can significantly reduce a child's cognitive development ability. As such, although various types of conventional hair growth agents have been tried to help hair growth with the purpose of promoting blood circulation in the scalp and supplying nutrients to the hair roots, these agents also have severe toxicity and side effects, and their effects are also insufficient.

On the other hand, when a peptide, a chain of amino acids, with an alopecia treatment effect is developed and used as an alopecia treatment agent, there is an advantage of high biocompatibility. Further, when a single peptide is used, its mechanism of action is clear compared to extracts containing various components, and it can easily be broken down and removed by proteases in the body after locally suppressing alopecia; unlike existing therapeutic agents, there are fewer concerns about the side effects.

As an example, the document Su Bin Hwang et al. “Hair-Growth-Promoting Effects of the Fish Collagen Peptide in Human Dermal Papilla Cells and C57BL/6 Mice Modulating Wnt/β-Catenin and BMP Signaling Pathways”, Int. J. Mol. Sci. 23, 2022, p. 2-15 describes the hair growth promoting effects of fish collagen-derived peptide Gly-Pro-Val-Gly-Pro-Ser (GPVGPS) and specifically discloses that this peptide can prevent or treat alopecia by reducing a growth inhibitory factor (TGF-β1).

As such, there is an urgent need to develop a new peptide preparation capable of effectively preventing alopecia and promoting hair growth with fewer side effects.

### RELATED ART

Su Bin Hwang et al. “Hair-Growth-Promoting Effects of the Fish Collagen Peptide in Human Dermal Papilla Cells and C57BL/6 Mice Modulating Wnt/β-Catenin and BMP Signaling Pathways”, Int. J. Mol. Sci. 5 23, 2022, p. 2-15

### SUMMARY

The present invention is directed to providing a scalp-derived peptide which is effective for treating alopecia.

The present invention is also directed to providing a composition for effectively preventing, treating, or ameliorating alopecia using the peptide described above.

In order to solve the above problems, the present inventors constructed a peptidomics-based low-molecular-weight peptide library through LC-MS² analysis of both non-alopecia (normal) scalp samples and alopecia scalp samples; from the library, they discovered candidate peptides with biomarker potential for distinguishing between a normal state and alopecia therefrom and conducted cell line experiments and human application tests to confirm that the above peptides were effective for alleviating alopecia symptoms, thereby completing the present invention.

According to an aspect of the present invention, there is provided a scalp-derived peptide consisting of an amino acid sequence of SEQ ID NO: 1 or SEQ ID NO: 2 with the ability to alleviate alopecia symptoms:

SEQ ID NO: 1:
RKLW
SEQ ID NO: 2:
RQLW

According to another aspect of the present invention, there is provided a pharmaceutical composition, comprising the peptide, for preventing or treating alopecia.

According to another aspect of the present invention, there is provided a cosmetic composition, comprising the peptide, for ameliorating alopecia.

According to another aspect of the present invention, there is provided a use of the peptide for preventing, treating or ameliorating alopecia.

According to another aspect of the present invention, there is provided a method for treating alopecia, the method including applying a therapeutically effective amount of the peptide to the head of a subject in need of alopecia treatment.

According to another aspect of the present invention, there is provided a method for ameliorating alopecia, the method including applying a cosmetically effective amount of the peptide to the head of a subject in need of alopecia amelioration.

The scalp-derived peptide consisting of the amino acid sequence of SEQ ID NO: 1 or SEQ ID NO: 2 according to the present invention increases the viability of cells that form hair follicles or are associated with hair formation, such as human keratinocytes and dermal papilla (DP) cells, which may help grow and maintain hair follicles.

In addition, the peptide of the present invention haves an activity of (1) suppressing the expression of an androgen receptor (AR) activated by binding to a dihydrotestosterone (DHT) hormone in cells associated with hair formation (for example, HaCaT and/or DP cells) and the activation of alopecia-related proteins Dickkopf-1 (DKK-1) and/or transforming growth factor beta (TGF-β) induced by activated AR, (2) increasing the expression of p-GSK-3β and/or β-catenin, which are Wnt signaling pathway proteins involved in hair follicle development and growth, (3) promoting the growth of cells by decreasing the expression ratio of Bax/Bcl-2 and decreasing the expression of cleaved caspase-3 and/or PARP to suppress the apoptosis of hair follicle-related cells, and (4) maintaining the survival of cells by increasing the expression levels of Cyclin D/CDK4 and/or Cyclin E/CDK2, which activate the G1 phase, and thus can be usefully used to prevent, treat or ameliorate alopecia, or promote hair growth.

Furthermore, the peptide of the present invention has an advantage of having higher biocompatibility and fewer problems such as a decrease in sexual function than existing therapeutic agents because a scalp-derived peptide is used as a single component and may be advantageous for skin permeation because it is an ultra-low-molecular-weight peptides (about 600 Da).

### BRIEF DESCRIPTION OF THE DRAWINGS

FIG. 1 schematically shows a method for screening for the scalp-derived peptides according to the present invention and shows a workflow for analyzing the bioactive peptides of scalp-derived samples using de novo sequencing. The abbreviation AA stands for androgenetic alopecia subjects and non-AA stands for non-androgenetic alopecia subjects.

FIG. 2 shows 10 peptide candidates each specific to the non-alopecia group (normal group) or alopecia group, which were discovered from a 16,475-peptide library constructed through de novo sequencing.

FIG. 3 shows the results of measuring cell viability after treating HaCaT cells, which are human keratinocytes, and dermal papilla (DP) cells with the peptides RKLW (SEQ ID NO: 1) and RQLW (SEQ ID NO: 2) according to the present invention.

FIG. 4 shows the cellular processes related to cell survival and proliferation in alopecia.

FIG. 5 shows the protein immunoblot (Western blot) results showing changes in the expression of an intracellular androgen receptor (AR) and its target proteins (DKK-1 and TGF-β) after treating human keratinocytes (HaCaT) and DP cells with the peptides RKLW (SEQ ID NO: 1) and RQLW (SEQ ID NO: 2) according to the present invention.

FIG. 6 shows the protein immunoblot (Western blot) results showing changes in the expression of intracellular Wnt signaling pathway proteins (p-GSK-3β and β-catenin) after treating human keratinocytes (HaCaT) and DP cells with the peptides RKLW (SEQ ID NO: 1) and RQLW (SEQ ID NO: 2) according to the present invention.

FIG. 7 shows the protein immunoblot (Western blot) results showing changes in the expression of intracellular mitochondrial apoptosis signaling pathway proteins (Bax, Bcl-2, Caspase-3, and PARP) after treating human keratinocytes (HaCaT) and DP cells with the peptides RKLW (SEQ ID NO: 1) and RQLW (SEQ ID NO: 2) according to the present invention.

FIG. 8 shows the protein immunoblot (Western blot) results showing changes in the expression of intracellular cell cycle marker proteins (Cyclin D1, CDK4, Cyclin E1, and CDK2) after treating human keratinocytes (HaCaT) and DP cells with the peptides RKLW (SEQ ID NO: 1) and RQLW (SEQ ID NO: 2) according to the present invention.

FIG. 9 shows a set of photographs of a hair tonic product produced using the peptides RKLW (SEQ ID NO: 1) and RQLW (SEQ ID NO: 2) according to the present invention.

FIG. 10 shows a phototrichogram image for measuring the hair count during a human application test of the hair tonic product according to the present invention and an example image of hair in a 1 cm² circle.

FIG. 11 shows the results of evaluating the total hair count as a primary efficacy evaluation variable during a human application test of the hair tonic product according to the present invention.

FIG. 12 shows the results of analyzing the variation (A) in the total hair count as a primary efficacy evaluation variable in the human application test of the hair tonic product according to the present invention.

### DETAILED DESCRIPTION

The following is a detailed description of the present invention.

### 1. Scalp-Derived Peptide Having Ability to Alleviate Alopecia Symptoms

The present invention provides a scalp-derived peptide that consists of an amino acid sequence of SEQ ID NO: 1 or SEQ ID NO: 2, with the ability to alleviate alopecia symptoms.

SEQ ID NO: 1:
RKLW
SEQ ID NO: 2:
RQLW

In the present invention, the peptide of SEQ ID NO: 1 and the peptide of SEQ ID NO: 2 may be used alone or in combination.

As shown in the examples to be described below, the present inventors confirmed that as a result of treating keratinocytes (human keratinocytes) and dermal papilla (DP) cells with a peptide having the amino acid sequence of SEQ ID NO: 1 or SEQ ID NO: 2, the viability of these cells was significantly increased, and as a result of conducting a human application test on patients with alopecia symptoms using a composition including the peptide, the total hair count was significantly increased. Therefore, the peptide having the amino acid sequence of SEQ ID NO: 1 or SEQ ID NO: 2 of the present invention may be used to prevent, treat or ameliorate alopecia.

Thus, in an aspect, the present invention provides a use of the peptide for preventing, treating or ameliorating alopecia.

As used herein, the term “peptide” refers to a polymer consisting of two or more amino acids linked by peptide bonds.

As used herein, the term “alopecia” refers to the absence of hair in a region where hair should be normally present and includes a phenomenon in which the hair count is reduced compared to the normal state. The alopecia includes both non-cicatricial alopecia and cicatricial alopecia depending on whether or not the hair follicles are destroyed. In an embodiment, alopecia may be selected from the group consisting of androgenetic alopecia, alopecia areata, telogen effluvium, traumatic alopecia, trichotillomania, pressure alopecia, anagen effluvium, pityriasis alopecia, alopecia syphilitica, seborrheic alopecia, symptomatic alopecia and congenital alopecia.

The peptide of the present invention may be obtained by various methods widely known in the art. As an example, the peptide of the present invention may be prepared using polynucleotide recombination and protein expression systems, or an in vitro synthesis method through chemical synthesis such as peptide synthesis, a cell-free protein synthesis method, and the like, but the preparation method thereof is not limited.

Further, in order to obtain better chemical stability, enhanced pharmacological characteristics (half-life, absorbability, potency, efficacy, and the like), altered specificity (for example, broad biological activity spectrum), and reduced antigenicity, a protecting group may be bound to the N-terminus or C-terminus of the peptide. For example, the protecting group may be an acetyl group, a fluorenyl methoxy carbonyl group, a formyl group, a palmitoyl group, a myristyl group, a stearyl group or polyethylene glycol (PEG), but it may be included without limitation as long as it is a component capable of modifying the peptide, particularly improving the stability of the peptide. The ‘stability’ refers not only to in vivo stability, which protects the peptide of the present invention from attack by proteolytic enzymes in vivo, but also to storage stability (for example, storage stability at room temperature).

The peptide of the present invention increases the viability of cells which form hair follicles or are associated with hair formation. Specifically, the peptide has an activity of inducing hair formation by increasing the viability of human keratinocytes and dermal papilla (DP) cells.

In addition, the peptide of the present invention has activity of (1) suppressing the expression of an androgen receptor (AR) activated by binding to a dihydrotestosterone (DHT) hormone in cells associated with hair formation (for example, HaCaT and/or DP cells) and the activation of alopecia-related proteins Dickkopf-1 (DKK-1) and/or transforming growth factor beta (TGF-β) induced by activated AR, (2) increasing the expression of p-GSK-3β and/or β-catenin, which are Wnt signaling pathway proteins involved in hair follicle development and growth, (3) promoting the growth of cells by decreasing the expression ratio of Bax/Bcl-2 and decreasing the expression of cleaved caspase-3 and/or PARP to suppress the apoptosis, and (4) maintaining the survival of cells by increasing the expression levels of Cyclin D/CDK4 and/or Cyclin E/CDK2, which activate the G1 phase, and thus can be usefully used to prevent, treat or ameliorate alopecia, or promote hair growth.

Furthermore, the peptide of the present invention has an advantage of having higher biocompatibility and fewer problems such as a decrease in sexual function than existing therapeutic agents because a scalp-derived peptide is used as a single component, and may be advantageous for skin permeation because it is an ultra-low molecular-weight peptides (about 600 Da).

### 2. Pharmaceutical Composition for Preventing or Treating Alopecia

Another aspect of the present invention provides a pharmaceutical composition that includes the peptide, for preventing or treating alopecia.

The pharmaceutical compositions of the present invention may include the peptide of SEQ ID NO: 1 and the peptide of SEQ ID NO: 2, alone or in combination.

As used herein, the term “prevention” refers to reducing the risk of developing a disease or disorder, and refers to any action that inhibits or delays the onset of a disease by preventing one or more clinical symptoms of the disease from progressing in a subject who has been exposed to the disease or is susceptible to the disease, but has not yet developed the disease or exhibited symptoms of the disease.

As used herein, the term “treatment” refers to the alleviation of a disease or disorder, and includes any action that ameliorates or beneficially alters the symptoms of a disease by inhibiting or reducing the progression of the disease or one or more clinical symptoms thereof.

The pharmaceutical composition of the present invention may include a therapeutically effective amount of the peptide, and may further include a pharmaceutically acceptable carrier. The term “therapeutically effective amount” refers to an amount sufficient to achieve the purpose of the present composition, which is to prevent or treat alopecia.

The pharmaceutical composition of the present invention may be an external skin preparation. The external skin preparation is a preparation which can be used by application to the outside of the skin, and when the pharmaceutical composition of the present invention is used as an external skin preparation, it may be applied to the scalp, specifically the scalp in an area where alopecia has occurred or the scalp in an area where hair growth is to be promoted.

In an embodiment, the external skin preparation may be selected from the group consisting of creams, gels, ointments, skin emulsions, skin suspensions, transdermal patches, drug-containing bandages, lotions, and combinations thereof. The external skin preparation may be appropriately mixed with components typically used in external skin preparations such as cosmetics or pharmaceuticals, such as an aqueous component, an oil-based component, a powder component, alcohols, a moisturizer, a thickener, an ultraviolet absorber, a whitening agent, a preservative, an antioxidant, a surfactant, a fragrance, a coloring agent, various skin nutrients, or a combination thereof, if necessary.

Hair to which the pharmaceutical composition of the present invention is applied includes hair roots and follicles of the head, hair on the head, eyelashes and eyebrows, beards, armpit hair, pubic hair, and all parts of the body where hair roots and hair follicles are found.

A suitable dosage of the pharmaceutical composition of the present invention may vary depending on the condition and body weight of a patient, the degree of disease, the form of drug, the administration route and period, and may be appropriately selected by a person skilled in the art.

### 3. Cosmetic Composition for Ameliorating Alopecia

Another aspect of the present invention provides a cosmetic composition that includes peptide for preventing or ameliorating alopecia.

The cosmetic compositions of the present invention may include the peptide of SEQ ID NO: 1 and the peptide of SEQ ID NO: 2, alone or in combination.

As used herein, the term “amelioration” refers to any action that ameliorates or benefits the symptoms of a disease or disorder.

The amelioration of alopecia may be achieved by eliminating the cause of alopecia, suppressing the progression of alopecia, suppressing hair from falling out, and promoting hair formation to promote hair growth.

The cosmetic composition may include a cosmetically effective amount of the peptide and a cosmetically acceptable carrier, and the cosmetically effective amount refers to an amount sufficient to achieve the above-described alopecia prevention or hair growth promoting efficacy.

The cosmetic composition of the present invention may contain a commonly used adjuvant, such as a hydrophilic or lipophilic gelling agent, a hydrophilic or lipophilic active agent, a preservative, an antioxidant, a solvent, a fragrance, a filler, a blocking agent, a pigment, an odor-absorbing agent, and a dye.

In an embodiment, the cosmetic composition of the present invention may be an external skin preparation formulation, and the external skin preparation formulation may be selected from the group consisting of a hair tonic, a hair cream, a hair lotion, a hair shampoo, a hair conditioner, a hair spray, a hair aerosol, a pomade, a powder gel, a hair pack, a hair treatment, an eyebrow hair growth product, an eyelash growth product and an eyelash nutrient.

Still another aspect of the present invention provides a method for treating alopecia, the method including applying a therapeutically effective amount of the peptide to the head of a subject in need of alopecia treatment.

Yet another aspect of the present invention provides a method for ameliorating alopecia, the method including applying a cosmetically effective amount of the peptide to the head of a subject in need of alopecia amelioration.

Hereinafter, the configuration and effects of the present invention will be described in more detail through examples. However, these examples are provided only for exemplarily describing the present invention, and the scope of the present invention is not limited by these examples.

### EXAMPLES

### Example 1: Screening for Scalp-Derived Bioactive Peptides

Through the LC-MS² analysis of non-alopecia (normal) scalp samples and alopecia scalp samples, a peptidomics-based low-molecular-weight peptide library was constructed to discover biologically active candidate substances.

Specifically, skin surface biopsy samples were obtained from non-alopecia subjects and alopecia subjects in the summer and winter (Table 1). To obtain the samples, a circular sticker with a diameter of 22 mm was used, and the sample was obtained from the forehead line.

TABLE 1
Selection of 25 normal subjects and alopecia subjects who
participated in summer/winter scalp sample collection

| Alopecia group | | | | | Non-alopecia group | | | | |
|---|---|---|---|---|---|---|---|---|---|
| | | | Management No. | | | | | Management No. | |
| No. | Name | Sex | Summer | Winter | No. | Name | Sex | Summer | Winter |
| 1 | K*H | F | A-V02 | A-V31 | 1 | L*E | M | B-V05 | B-V35 |
| 2 | L*K | M | A-V06 | A-V56 | 2 | J*J | F | B-V06 | B-V36 |
| 3 | J*K | F | A-V12 | A-V50 | 3 | L*R | M | B-V07 | B-V47 |
| 4 | K*J | F | A-V13 | A-V51 | 5 | Y*J | F | B-V13 | B-V39 |
| 5 | M*S | F | A-V14 | A-V36 | 6 | J*H | F | B-V14 | B-V57 |
| 6 | L*M | F | A-V17 | A-V43 | 7 | K*H | F | B-V15 | B-V40 |
| 7 | P*H | F | A-V18 | A-V33 | 8 | N*Y | F | B-V16 | B-V48 |
| 8 | L*M | M | A-V20 | A-V49 | 9 | N*W | M | B-V17 | B-V52 |
| 9 | K*G | F | A-V21 | A-V42 | 10 | P*J | M | B-V21 | B-V46 |
| 10 | J*K | M | A-V23 | A-V58 | 11 | J*R | F | B-V26 | B-V54 |
| 11 | J*H | M | A-V25 | A-V57 | 12 | K*J | F | B-V29 | B-V59 |
| 12 | J*S | F | A-V26 | A-V40 | | | | | |
| 13 | H*M | F | A-V27 | A-V41 | | | | | |
| 14 | L*E | F | A-V29 | A-V53 | | | | | |

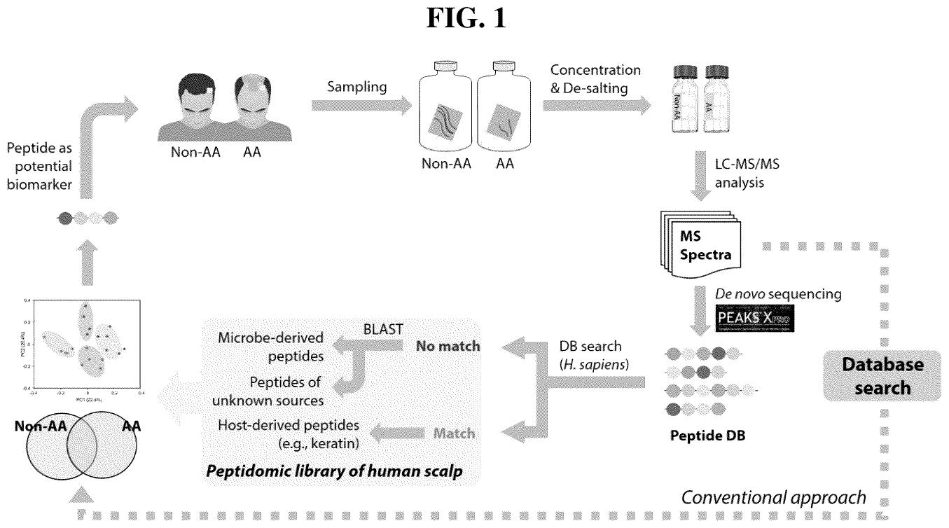

In order to discover peptides with biomarker potential for distinguishing between a normal state and alopecia based on peptidomics analysis, a workflow for bioactive peptide analysis of scalp-derived samples using de novo sequencing was designed (FIG. 1).

A total of 50 samples were de-salted and concentrated, and peptide MS/MS spectra were obtained through LC-MS² (Eksigent nanoLC/Thermo LTQ-XL) analysis. The device conditions used for the LC-MS² analysis are shown in the following Table 2.

TABLE 2
Information on LC-MS² instrument

| | |
|---|---|
| LC instrument | Eksigent nanoLC |
| MS instrument | Thermo LTQ-XL |
| Ion source | ESI (nano-spray) |
| Fragmentation mode | CID |
| MS scan mode | Linear Ion Trap |
| MS/MS scan mode | Linear Ion Trap |

De novo sequencing was performed on the obtained spectra using PEAKS Studio 10.6 software, and a 16,475 peptide library with a de novo score of 50 or higher was constructed. The conditions used for PEAKS Studio analysis are shown in the following Table 3.

TABLE 3
PEAKS Studio analysis conditions

| Software | PEAKS Studio 10.6 |
|---|---|
| Parent m/z tolerance | 4.0 Da |
| Fragment m/z tolerance | 1.0 Da |
| Enzyme | No enzyme |
| Modifications | (Variable) M <ox> |
| Maximum variable PTMs | 2 |

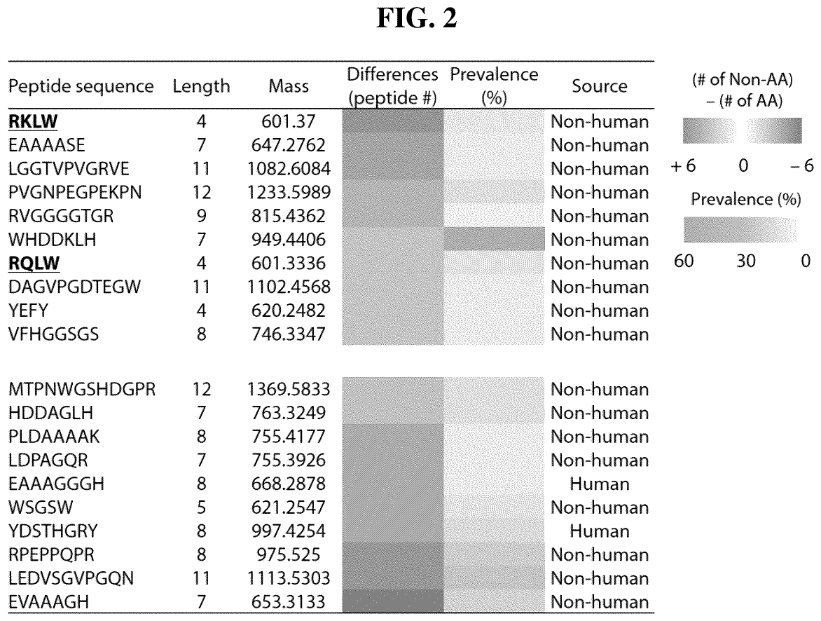

Among the above peptide library, about 10% were aligned to proteins derived from the Homo sapiens proteome database (UniProt ID: UP000005640), and the remaining 90% were estimated to be peptides derived from microorganisms or other foreign substances. As a result, 10 peptide candidates each specific to the normal group or the alopecia group were discovered (FIG. 2), and sequences specific to the non-alopecia group (RKLW and RQLW) were synthesized. Information on RKLW (SEQ ID NO: 1) and RQLW (SEQ ID NO: 2) is shown in the following Table 4.

TABLE 4
Information on sequences specific to non-alopecia group (RKLW and
RQLW)

| Sequence | | |
|---|---|---|
| (Hydrophobic/ | | |
| acidic/basic) | RKLW | RQLW |
| Structure http:// pepdraw.com | 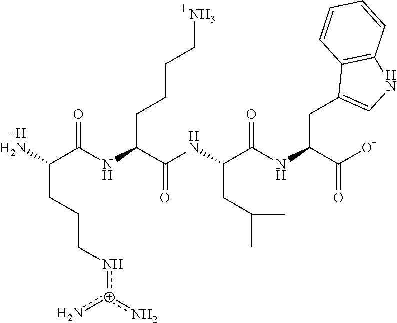 | 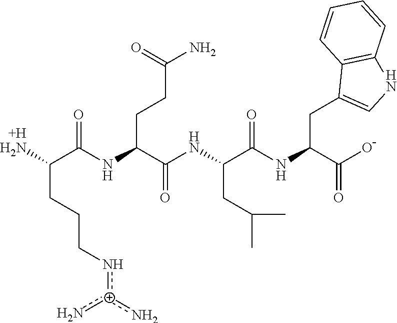 |
| Length | 4 | 4 |
| Mass | 601.75 | 601.71 |
| Net charge | +2 | +1 |
| (at pH 7) | | |
| Hydrophobicity | Hydrophobic: 50% | Hydrophobic: 50% |
| | Acidic: 0% | Acidic: 0% |
| | Basic: 50% | Basic: 25% |
| | Neutral: 0% | Neutral: 25% |

### Example 2: Verification of Ability of Scalp-Derived Peptides to Alleviate Alopecia Symptoms

(1) Analysis of Skin Cell Viability According to Presence or Absence of Treatment with Peptides (RKLW and RQLW)

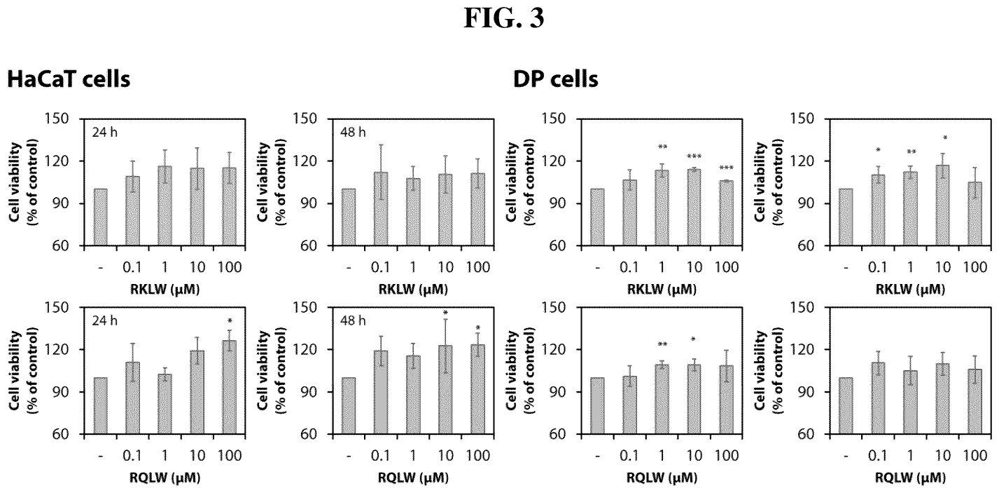

In order to evaluate the efficacy of peptide treatment (RKLW and RQLW) to promote skin cell proliferation, human keratinocytes and dermal papilla (DP) cells were treated with RKLW and RQLW at 0.1, 1, and 10 μM. After being cultured for 24 and 48 hours, cells were treated with Cell Counting Kit-8 (CCK-8) to measure cell viability, and the results are shown in FIG. 3.

As shown in FIG. 3, the cell viability of HaCaT cells was increased by RKLW and RQLW treatment, but RKLW did not show statistical significance, and RQLW significantly increased cell viability at concentrations of 100 μM or higher after 24 hours and 10 μM or higher after 48 hours.

For dermal papilla cells, treatment with RKLW and RQLW at concentrations of 1 and 10 μM for 24 hours significantly increased cell viability. In addition, when treated for 48 hours, RKLW significantly increased cell viability at a treatment concentration of 10 μM or less, but RQLW showed no significant difference.

As a result, RKLW and RQLW significantly increased the cell viability of dermal papilla cells and/or HaCaT cells, which may help the growth and maintenance of hair follicles.

(2) Analysis of Skin Cell Responses with and without Treatment with Peptides (RKLW and RQLW)

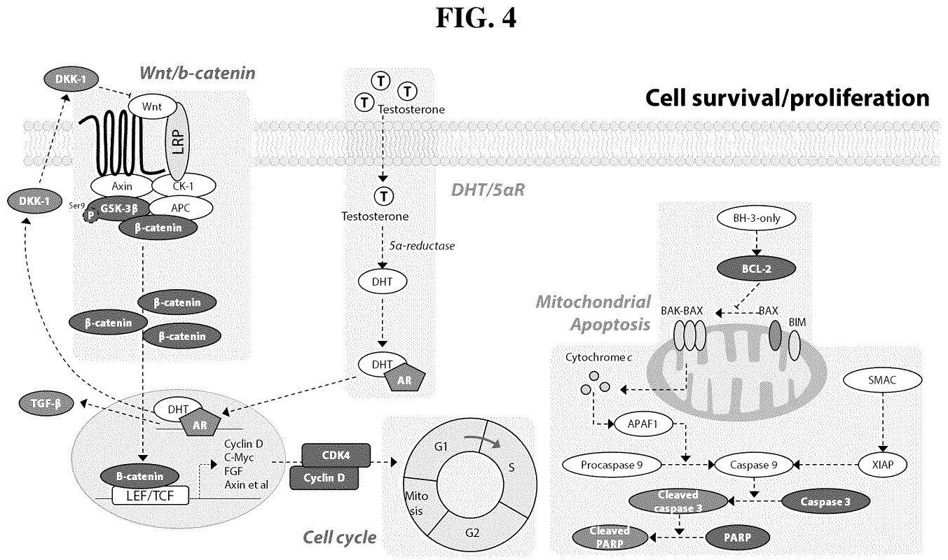

The expression and phosphorylation of protein markers involved in alopecia-related signal transduction pathways were evaluated after treatment with peptides (RKLW and RQLW). The cellular processes related to cell survival and proliferation metabolic process in alopecia are shown in FIG. 4.

DHT-AR and Wnt/beta-catenin pathway: An androgen receptor (AR) is a protein that binds to dihydrotestosterone (DHT), a representative inducer of androgenetic alopecia, and when the AR is activated by binding to DHT, Dickkopf-1 (DKK-1) and transforming growth factor beta (TGF-β) are expressed and promote alopecia. In addition, DKK-1, which is expressed by the activation of the AR, suppresses the Wnt signaling pathway involved in the development and growth of hair follicles, and TGF-β induces alopecia by suppressing the growth of hair follicle cells.

Specifically, the expressions of proteins involved in major signaling pathways related to hair growth were analyzed when HaCaT and DP cells were treated with RKLW and RQLW at 0.1, 1, and 10 μM and cultured for 24 hours.

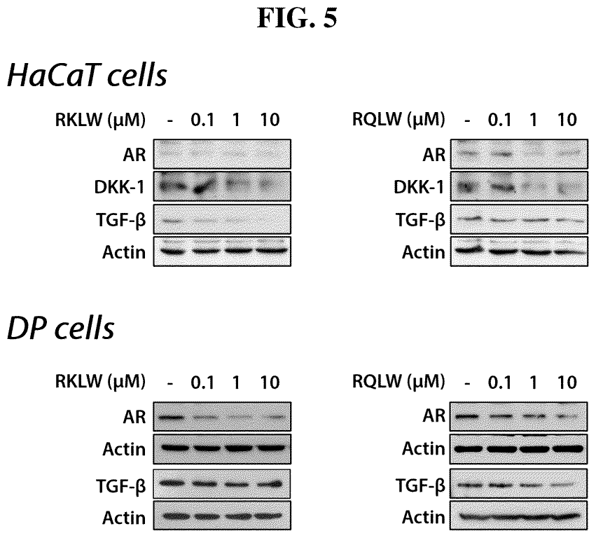

The protein immunoblot (Western blot) results of the expression of the AR and its target proteins (DKK-1 and TGF-β) in HaCaT and DP cells are shown in FIG. 5. As shown in FIG. 5, no significant change in AR expression was observed when HaCaT cells were treated with RKLW or RQLW, but the expression levels of DKK-1 and TGF-β, target proteins of AR, were both decreased. When DP cells were treated with RKLW or RQLW, the expression levels of both the AR and its target protein TGF-β was reduced.

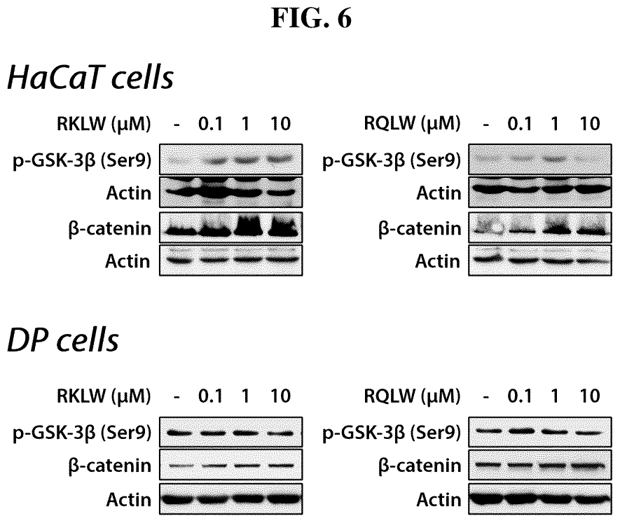

Furthermore, the protein immunoblot (Western blot) results showing differential expressions and phosphorylation of proteins involved in Wnt signaling pathway in HaCaT and DP cells are shown in FIG. 6. As shown in FIG. 6, when HaCaT cells were treated with RKLW or RQLW, the expression levels of p-GSK-3β and β-catenin increased, confirming that the Wnt signaling pathway was activated. When DP cells were treated with RKLW or RQLW, the expression of p-GSK-3β was not increased, but the expression of β-catenin was increased, meaning that in DP cells, RKLW or RQLW accumulates β-catenin through a pathway other than GSK-3β to activate Wnt signaling.

Mitochondrial apoptosis: Bax is a pro-apoptotic protein that promotes apoptosis, and Bcl-2 is a pro-survival protein that suppresses apoptosis; the balance between these two proteins is important in regulating apoptosis.

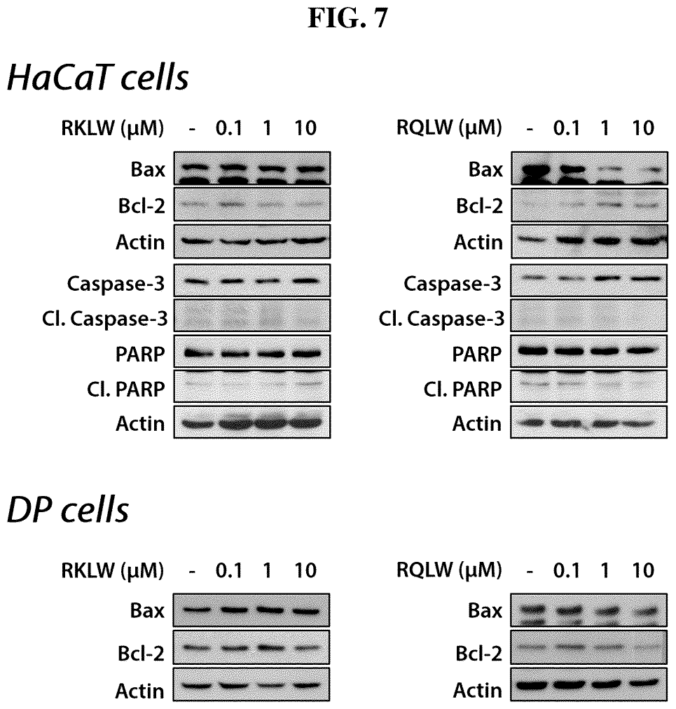

Specifically, the expressions of proteins involved in mitochondrial apoptotic signaling pathways were analyzed when HaCaT and DP cells were treated with RKLW and RQLW at 0.1, 1, and 10 μM and cultured for 24 hours, and the results are shown in FIG. 7.

As shown in FIG. 7, RQLW tended to decrease the expression ratio of Bax/Bcl-2 and the protein levels of cleaved caspase-3 and PARP in HaCaT cells. Through this, it can be seen that RQLW treatment showed an effect of suppressing apoptosis. However, RKLW showed a relatively insignificant effect. The above results are consistent with the cell viability analysis results (FIG. 3), indicating that RQLW can promote the growth of HaCaT cells by suppressing the apoptosis mechanism. In the case of DP cells, both RKLW and RQLW were unable to regulate the expression ratio of Bax/Bcl-2, and these results indicate that RKLW and RQLW do not affect the apoptosis mechanism of DP cells.

Cell cycle: maintaining the G1 phase of the cell cycle of scalp skin cells plays an important role in maintaining scalp health.

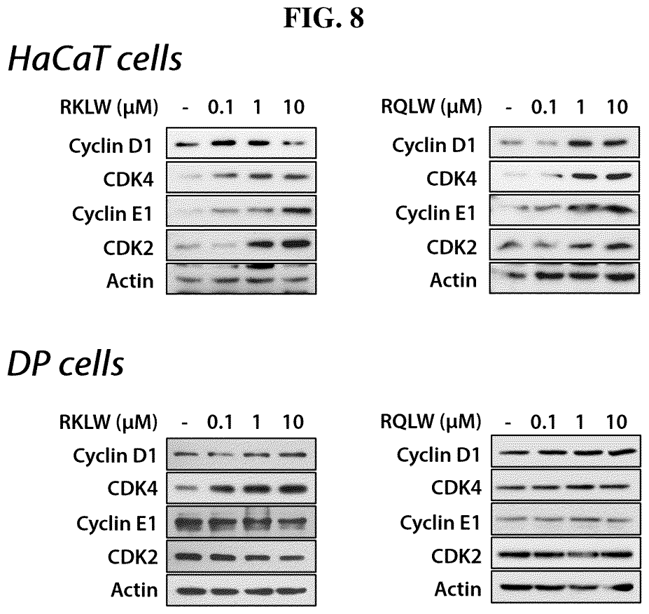

Specifically, the expressions of Cyclin D/CDK4 and Cyclin E/CDK2, which activate the G1 phase, were analyzed when HaCaT and DP cells were treated with RKLW and RQLW at 0.1, 1, and 10 UM and cultured for 24 hours, and the results are shown in FIG. 8. As shown in FIG. 8, RKLW and RQLW increased the expression levels of Cyclin D/CDK4 and Cyclin E/CDK2, which activate the G1 phase, in HaCaT cells, indicating that RKLW and RQLW can maintain the survival of cells. In the case of DP cells, RKLW and RQLW increased the expression level of Cyclin D/CDK4, but did not affect the expression level of Cyclin E/CDK2. These results indicate that RKLW and RQLW can maintain the survival and growth of cells by specifically activating the early G1 phase in DP cells.

### Example 3: Cosmetic Composition Comprising Scalp-Derived Peptide as Active Ingredient and its Efficacy

### (1) Production of Hair Tonic Comprising Peptide as Active Ingredient

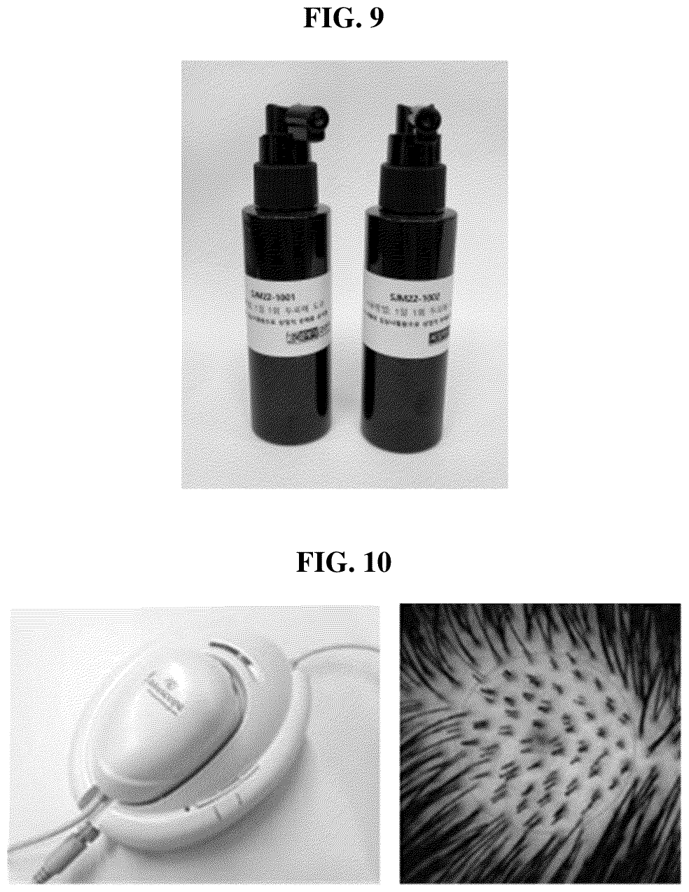

A hair tonic was produced using a cosmetic composition including peptides (RKLW and RQLW) as active ingredients. Specifically, the product formulation was selected to be a leave-on hair tonic such that the peptide could be sufficiently absorbed into the scalp or hair. Test products were produced with 0.005% RKLW and 0.005% RQLW so that a final peptide concentration was 0.01%, and control products were produced with the same formulation except for peptide (FIG. 9). All ingredients of the test and control products are shown in Table 5.

TABLE 5
All ingredients of test and control products

| Product | No. | Ingredient name | INCI name |
|---|---|---|---|
| Test | 1 | Purified water | Water |
| product | 2 | Ethyl alchol | Alcohol |
| | 3 | 1.3-butylene glycol | Butylene Glycol |
| | 4 | Peppermint oil | Mentha piperita |
| | | | (peppermint) oil |
| | 5 | Citric acid | Citric acid |
| | 6 | Betaine | Betaine |
| | 7 | Sodium citrate | Sodium citrate |
| | 8 | Peptide 0.01% (RKLW 0.005%, | |
| | | RQLW 0.005%) | |
| Control | 1 | Purified water | Water |
| product | 2 | Ethyl alchol | Alcohol |
| | 3 | 1.3-butylene glycol | Butylene Glycol |
| | 4 | Peppermint oil | Mentha piperita |
| | | | (peppermint) oil |
| | 5 | Citric acid | Citric acid |
| | 6 | Betaine | Betaine |
| | 7 | Sodium citrate | Sodium citrate |

### (2) Primary Irritation Test of Hair Tonic

In accordance with the Korea Ministry of Food and Drug Safety Notification No. 2020-131, a human skin primary irritation test was conducted on more than 30 men and women. The information on the subjects participating in the primary irritation test is shown in the following Table 6.

TABLE 6
Information on subjects for
primary irritation test

| | |
|---|---|
| No. of subjects | 32 |
| participating in test (N) | |
| Those who dropped out | 2 |
| during test (N) | (Violation of |
| | selection criteria) |
| No. of final subjects | 30 |
| who completed test (N) | |
| Average age ± | 40.9 ± 10.95 |
| Standard deviation | years old |
| Sex (N) | M: 3, F: 27 |

In the test, a test substance was instilled on the back of a subject and the patch was kept closed for 24 hours, and then irritation was checked; skin irritation by the test substance was visually evaluated twice: 30 minutes and 24 hours after patch removal. As a result of determining the presence or absence of skin safety by combining these factors, both the test and control products had zero skin reactivity and were determined to be non-irritating (Table 7).

TABLE 7
Primary irritation test determination results

| Requested | No. of | Skin reactivity | | Average skin | Deter- |
|---|---|---|---|---|---|
| product name | responders | 30 min | 24 hr | reactivity* | mination |
| Peptide-containing | 0 | 0 | 0 | 0 | Non-irritant |
| hair tonic | | | | | |
| Control hair tonic | 0 | 0 | 0 | 0 | Non-irritant |
| Blank (Control) | 0 | 0 | 0 | 0 | Non-irritant |
| Distilled water | 0 | 0 | 0 | 0 | Non-irritant |
| (Negative control) | | | | | |

*Average skin reactivity = (Sum of skin reactivities)/2

### (3) Human Study Using Hair Tonic on Androgenic Alopecia Patients

In accordance with the Korea Ministry of Food and Drug Safety guidelines (Human study guidelines for cosmetics useful for alleviating alopecia symptoms, Guidebook-0760-02, July 2018), a randomized, double-blind, control comparison simple human study was conducted for 12 weeks (Table 8). The information on the subjects participating in the human study is shown in the following Table 8.

TABLE 8
Information on subjects for human study

| | |
|---|---|
| No. of subjects | 48N |
| participating in test | |
| Those who dropped | 0N |
| out during test | |
| No. of final subjects | 48N |
| who completed test | |
| Sex | F: 48N |
| Average age ± | 40.9 ± 9.53 |
| Standard deviation | years old |

An alopecia grade was determined by a dermatologist for a total of 48 subjects in accordance with the Ministry of Food and Drug Safety guidelines (Guidelines for human study of cosmetics useful for alleviating alopecia symptoms, Guidebook-0760-02, July 2018), and then subjects who met the inclusion criteria for selection were selected as shown in the following Table 9.

TABLE 9
Alopecia grade classification of subjects

| Alopecia classification | | |
|---|---|---|
| method | BASP | Ludwig |
| No. of subjects (N) | 42 | 42 |
| | 5 | 5 |
| | 1 | 1 |

A total of 48 subjects were evaluated for the primary endpoint (total hair count) and secondary endpoint (subject survey evaluation) before using the product (week 0) and after 6 and 12 weeks of product use. In order to confirm whether randomization was carried out effectively, comparisons were made between groups with respect to the subject's age, the hair count before using the product (week 0), which is the primary endpoint, and scalp and hair characteristics collected through subject survey evaluation before using the product (week 0), and it was confirmed that randomization was effectively carried out as the significance probability exceeded 0.1 and the groups were randomly allocated.

### (4) Evaluation of Total Hair Count as Primary Endpoint

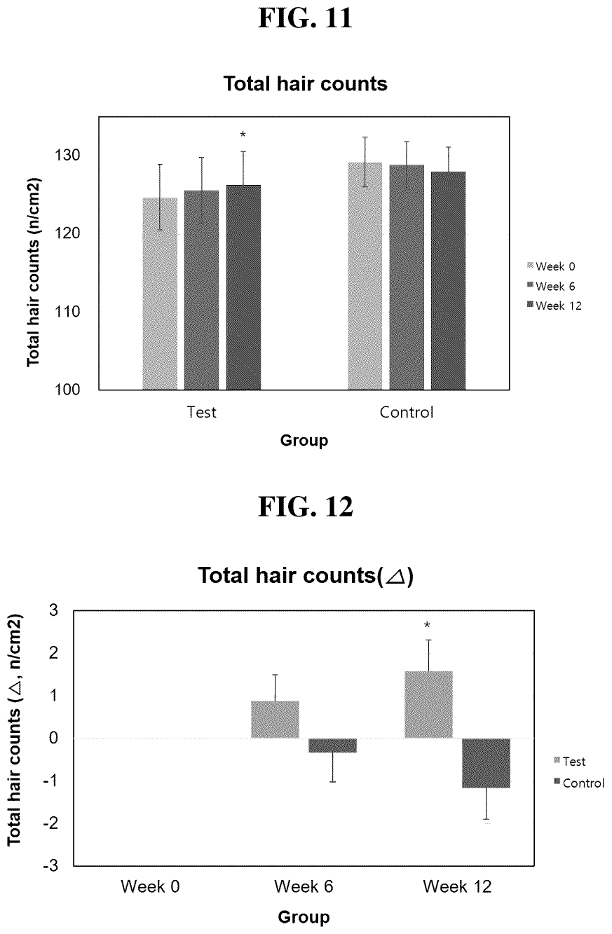

Before (week 0) and after 6 and 12 weeks of using the product, the hair in a 1 cm² circle was uniformly removed around a dot tattoo on the alopecia area of a subject's scalp, and images were photographed using Folliscope 5.0 (LeadM, Korea), an imaging device, at 14× magnification. The total hair counts (number/cm²) were analyzed using a phototrichogram (FIG. 10), and the results are shown in FIG. 11. It could be confirmed that the total hair count in the test group significantly increased after 12 weeks of using the product compared to before using the product (week 0) (p<0.05). In contrast, no statistically significant differences were observed in the control.

In addition, a statistically significant (p<0.05) differences were observed in the variation in hair count (4) between the groups before (week 0) and after 12 weeks of using the product (FIG. 12).

### (5) Subject Survey Evaluation as Secondary Endpoint

A survey evaluation was conducted on the efficacy and usability of the product after 6 and 12 weeks of product use. For the results of the survey evaluation regarding the efficacy of the product, the responses of the test group and the control were compared (Table 10), and a positive response rate (Table 11) was analyzed.

TABLE 10
Results of comparison between survey
evaluation groups regarding efficacy

| | | p-value | |
|---|---|---|---|
| Group | Item | Week 6 | Week 12 |
| Test vs. | Amelioration of alopecia area | 0.475 | 0.085 |
| Control | Hair on top of head | 0.383 | 0.224 |
| | becomes fuller | | |
| | Improved crown parting | 1.000 | 0.033* |
| | Hairline becomes fuller | 0.233 | 0.024* |
| | Improvement of hairline | 0.302 | 0.100 |
| | Reduction in hair | 0.634 | 0.058 |
| | counts falling out | | |
| | Hair satisfaction | 0.748 | 0.379 |

Test vs. Control, Mann-Whitney U test, Significance:
*p < 0.05

TABLE 11
Positive response rate for survey evaluation regarding efficacy¹

| | Test (N = 24) | | | | Control (N = 24) | | | |
|---|---|---|---|---|---|---|---|---|
| | Week 6 | | Week 12 | | Week 6 | | Week 12 | |
| Item | N² | %³ | N² | %³ | N² | %³ | N² | %³ |
| Amelioration of alopecia | 14 | 58.33 | 17 | 70.83 | 11 | 45.83 | 14 | 58.33 |
| area | | | | | | | | |
| Hair on top of head | 13 | 54.17 | 18 | 75.00 | 10 | 41.67 | 14 | 58.33 |
| becomes fuller | | | | | | | | |
| Improved crown parting | 12 | 50.00 | 18 | 75.00 | 11 | 45.83 | 12 | 50.00 |
| Hairline becomes fuller | 15 | 62.50 | 17 | 70.83 | 11 | 45.83 | 9 | 37.50 |
| Improvement of hairline | 14 | 58.33 | 17 | 70.83 | 11 | 45.83 | 12 | 50.00 |
| Reduction in hair counts | 13 | 54.17 | 18 | 75.00 | 11 | 45.83 | 11 | 45.83 |
| falling out | | | | | | | | |
| Hair satisfaction | 14 | 58.33 | 17 | 70.83 | 16 | 66.67 | 16 | 66.67 |

¹Positive response rate (%): Percentage of subjects who answered in the positive (scale 5-7) out of all subjects
²N(Frequency): No. of subjects who answered in the positive (scale 5-7) out of all subjects
³%(Percentage): (No. of subjects who answered in the positive (scale 5-7)/(Total number of subjects)*100%

After 12 weeks of product use, a statistically significant difference between the test group and the control could be confirmed in positive responses to the items “improved crown parting and “hairline becomes fuller.” (p<0.05).

Furthermore, after using the product, scalp safety was evaluated through interviews and observations by the test personnel at all evaluation points, and as a result, no special adverse reactions caused by the test product were observed during the test period (Table 12).

TABLE 12
Adverse reaction symptoms

| Abnormal response symptoms | | Week 6 | Week 12 |
|---|---|---|---|
| Subjective | Itchy | 0 | 0 |
| sensation of | It hurts like a | 0 | 0 |
| irritation | needle prick | | |
| | Burning | 0 | 0 |
| | Tingling | 0 | 0 |
| | Stiff | 0 | 0 |
| | Others | 0 | 0 |
| Objective | Erythem | 0 | 0 |
| irritation | Edema | 0 | 0 |
| | Papule | 0 | 0 |
| | Others | 0 | 0 |

| | | |
|---|---|---|
| No. of subjects with | 0 | 0 |
| abnormal responses | | |

1. A scalp-derived peptide consisting of amino acid sequences of SEQ ID NO: 1 or SEQ ID NO: 2 with the ability to alleviate alopecia symptoms:
SEQ ID NO: 1:
RKLW;
and
SEQ ID NO: 2:
RQLW.

2. The scalp-derived peptide of claim 1, wherein the peptide of SEQ ID NO: 1 and the peptide of SEQ ID NO: 2 are used alone or in combination.

3. The scalp-derived peptide of claim 1, wherein the alopecia is selected from the group consisting of androgenetic alopecia, alopecia areata, telogen effluvium, traumatic alopecia, trichotillomania, pressure alopecia, anagen effluvium, pityriasis alopecia, alopecia syphilitica, seborrheic alopecia, symptomatic alopecia and congenital alopecia.

4. The scalp-derived peptide of claim 1, wherein the peptide increases the viability of human keratinocytes and dermal papilla (DP) cells.

5. The scalp-derived peptide of claim 1, wherein the peptide suppresses the expression of an androgen receptor (AR), Dickkopf-1 (DKK-1), and/or transforming growth factor beta (TGF-β), which are alopecia-related proteins, in cells associated with hair formation.

6. The scalp-derived peptide of claim 1, wherein the peptide increases the expression of p-GSK-3β and/or β-catenin, which are Wnt signaling pathway proteins involved in the development and growth of hair follicles, in cells associated with hair formation.

7. The scalp-derived peptide of claim 1, wherein the peptide promotes the growth of cells by decreasing the expression ratio of Bax/Bcl-2 and decreasing the expression of cleaved caspase-3 and/or PARP to suppress apoptosis in cells associated with hair formation.

8. The scalp-derived peptide of claim 1, wherein the peptide maintains the survival of cells by increasing the expression levels of Cyclin D/CDK4 and/or Cyclin E/CDK2, which activate the G1 phase in cells associated with hair formation.

9. A method of preventing or treating alopecia, the method comprising administering a pharmaceutical composition containing the scalp-derived peptide of claim 1.

10. The method of claim 9, wherein the peptide of SEQ ID NO: 1 and the peptide of SEQ ID NO: 2 are used alone or in combination.

11. The method of claim 9, wherein the alopecia is selected from the group consisting of androgenetic alopecia, alopecia areata, telogen effluvium, traumatic alopecia, trichotillomania, pressure alopecia, anagen effluvium, pityriasis alopecia, alopecia syphilitica, seborrheic alopecia, symptomatic alopecia and congenital alopecia.

12. The method of claim 9, wherein the pharmaceutical composition is an external skin preparation.

13. The method of claim 12, wherein the external skin preparation is selected from the group consisting of a cream, a gel, an ointment, a skin emulsion, a skin suspension, a transdermal patch, a drug-containing bandage, a lotion, and a combination thereof.

14-18. (canceled)


SEQUENCE LISTING (2 sequences)

SEQ ID NO: 1 - protein, 4 aa, Homo sapiens
RKLW

SEQ ID NO: 2 - protein, 4 aa, Homo sapiens
RQLW